(19) United States
(12) Patent Application Publication
(10) Pub. No.: US 2004/0202570 A1
(43) Pub. Date: Oct. 14, 2004

(54) System for disinfection of buildings using ozone

(76) Inventor: Shyam K. Nadkarni, Woodmere, NY (US)

Correspondence Address: PATREA L. PABST, PABST PATENT GROUP LLP, 400 COLONY SQUARE, SUITE 1200, ATLANTA, GA, 30361, US

(21) Appl. No.: 10/412,777

(22) Filed: Apr. 11, 2003

(51) Int. Cl. A61L 2/00; B32B 5/02

(52) U.S. Cl. 422/28; 422/29; 422/24; 422/2; 422/186.07; 250/455.11; 422/123; 422/121

(57) ABSTRACT

A process for disinfecting an area such as a building or mail processing system including the steps of connecting a sealed area with a disinfecting system capable of generating ozone, and circulating air including ozone within the sealed area and the system for a desired time. By selecting the concentration of ozone and time period to kill spores, while minimizing damage to materials from the ozone, one is able to decontaminate the building rapidly and inexpensively. The process may further include the step of illuminating interior spaces and/or the air circulated from the area with ultraviolet light, and/or the step of adjusting the relative humidity to a desired level. A system for disinfecting an area includes a generator for generating ozone, a detector for measuring the concentration of ozone, and a regulator for controlling the generator, wherein a disinfecting concentration of ozone can be achieved and maintained within the area.

### BACKGROUND OF THE INVENTION

The present invention relates to processes and systems for disinfecting with ozone.

Environmental pollution is becoming a more and more serious problem to modern society. Exposure to environmental pollutants pose varying degrees of adverse health effects. Recent scientific studies indicate, however, what people are breathing in residence or other buildings can be more polluted than the outside in the industrialized cities. About 30 percent of new and remodeled buildings worldwide may be subject to excessive indoor pollution. These problems are often a result of poor building design or occupant activities.

Recent attacks from bioterrorists make the situation far worse. There have been several reports that the mail has been contaminated with anthrax, which may have led to the death of individuals exposed to the contaminated mail, as well as lengthy and expensive decontaminations and cleanups of buildings in which the mail was sorted or distributed.

Anthrax is an infection caused by the bacteria Bacillus anthracis, a gram positive bacteria. Although it's most commonly found in grazing animals like sheep, pigs, cattle, horses, and goats, anthrax can sometimes infect humans. In the environment, anthrax can form spores that can live in the soil for years. People can become infected by coming in contact with these spores through a break in their skin (such as a cut, scratch, or rash), by eating contaminated food (usually meat), or by inhaling the spores.

There are three main types of anthrax:

cutaneous (skin) anthrax, which can occur if someone handles contaminated animals or animal products (especially animal hides) while they have a cut, abrasion, or rash on the skin

intestinal anthrax, which can occur if someone eats contaminated meat pulmonary (inhaled) anthrax, which is extremely rare but can occur if someone breathes anthrax spores—usually found in the dust kicked up by animals—all the way down into the lungs

More than 95% of anthrax cases in the world are from cutaneous anthrax. This is the least dangerous form. Intestinal anthrax is much less common, but it can make a person much sicker.

Symptoms vary depending on the type of anthrax:

Cutaneous anthrax usually involves skin sores that may turn black after a few days. (The name “anthrax” comes from the Greek word for coal and refers to these sores.)

Intestinal anthrax symptoms include severe abdominal pain, nausea, vomiting, severe diarrhea, and bleeding from the gastrointestinal tract.

Pulmonary anthrax usually begins with flu-like symptoms but, if untreated, can rapidly turn into severe pneumonia.

It usually takes less than 7 days for symptoms of skin and intestinal anthrax to appear, but symptoms can appear as early as 48 hours after the lungs have been exposed to anthrax spores.

There is currently no fast, efficient and relatively inexpensive way to safely decontaminate large buildings, especially those exposed to spores, for example, anthrax spores.

### BRIEF SUMMARY OF THE INVENTION

A process for disinfecting an area such as a building or mail processing system including the steps of connecting a sealed area with a disinfecting system capable of generating ozone, and circulating air including ozone within the sealed area and the system for a desired time. By selecting the concentration of ozone and time period to kill spores, while minimizing damage to materials from the ozone, one is able to decontaminate the building rapidly and inexpensively. The process may further include the step of illuminating interior spaces and/or the air circulated from the area with ultraviolet light, applying ultrasonic energy or negative ionization, and/or the step of adjusting the relative humidity to a desired level. A system for disinfecting an area includes a generator for generating ozone, a detector for measuring the concentration of ozone, and a regulator for controlling the generator, wherein a disinfecting concentration of ozone can be achieved and maintained within the area.

### BRIEF DESCRIPTION OF THE DRAWINGS

FIG. 1 is a flow diagram of the process and equipment for disinfecting the air in a building which may have been contaminated with a biohazard such as anthrax spores.

### DETAILED DESCRIPTION OF THE INVENTION

A process has been developed for disinfection of large volumes of air in an office building, postal office or supply or warehouse, utilizing ozone, high temperature/incineration/filtration, and another means such as ultraviolet light, to disin-fect the air. A particular advantage of the system is that it is portable and can be transported to the site and used for any type of building or vessel such as a ship, with complete disinfection typically within 24 hours.

Ozone

Ozone (O₃) is a molecule composed of three oxygen atoms arranged in a V-shape. It is an allotrope of oxygen (O₂), i.e., it is composed of the same oxygen atoms combined in a different way. Ozone is one of the most powerful and useful oxidants. The high chemical reactivity of ozone arises from its unstable electron configuration that seeks electrons from other molecules. When reacting with other molecules, ozone oxidizes the molecules and is converted into oxygen. Through oxidization, ozone can be used to eliminate bacteria, viruses, mold, mildew, spores, cysts, yeast, fungus, smoke and etc., at least under laboratory conditions (see, for example, Ishizaki, et al., J. Appl. Bacterial. 60(1):67-72 (1986); Rickloff, Appl. Environ. Microbiol. 53(4):583-686 (1987); Tan, et al., Jikken Bobutsu 39(3):371-376 (1990) Driedger, et al., Water. Res. 35(12):2950-2960 (2001)). In general, the conditions used under laboratory conditions for killing of spores were extreme, for example, 200 ppm ozone at 60% relative humidity (RH) for a period in excess of 200 minutes, more preferably over 1000 minutes. Using higher humidity or ozone saturated water provided better results, but clearly is not practical for disinfecting office buildings or mail handling systems which are full of paper.

In addition to oxidation, ozone by itself decays back into oxygen due to its chemical instability. The natural process is speeded up by the presence of walls, carpets, furniture etc. The half life of ozone is at most within 30 minutes.

Ozone has been demonstrated to be a highly effective biocide in water, while the use of ozone in air has not been reported as widely as its use in water. Nevertheless, disinfection with gaseous ozone has been demonstrated to be superior to other disinfecting approaches, e.g., formaldehyde vaporization, because of convenience, insignificant inhalation of the disinfectant by people, and very rapid expulsion of the gas after ventilation. For example, gaseous ozone has been used to disinfect clean rooms, as reported by Masaoka, et al. Appl. Environ. Microbiol. 43(3): 509-513 (March 1982) but at a concentration that was caustic to surfaces such as rubber (80 mg/square meter for 72 hours), conditions that clearly would not be useful for disinfecting office buildings, airplanes or other facilities containing paper, furniture, computers, etc.

A research proposal to treat bacteria generally in ventilation air is described at http://www.bio.psu.edu/people/faculty/whittam/research/two.htm, but there is no indication of the proposal being funded or any solutions proposed on how ventilation air can actually be disinfected.

Systems for Disinfecting Buildings, Mail Handling Systems and Airplanes

Systems made up of interconnected modules of components have been designed for disinfecting all areas accessible to a gas mixture in a building, enclosure, warehouse, ship, or other enclosed and accessible structure. Disinfecting gas mixtures consist of a controlled mixture of ozone, oxygen or air, moisture (in the form of relative humidity) and ultra-violet light that is continuously moved from the treated area through various system modules until the air has been treated with the desired concentration of ozone, humidity, and light, to disinfect or destroy the specific bacteria, virus, molds, or spores in issue and/or all of the Ozone is depleted. The exhaust air is incinerated in a kiln fitted with an emission control system.

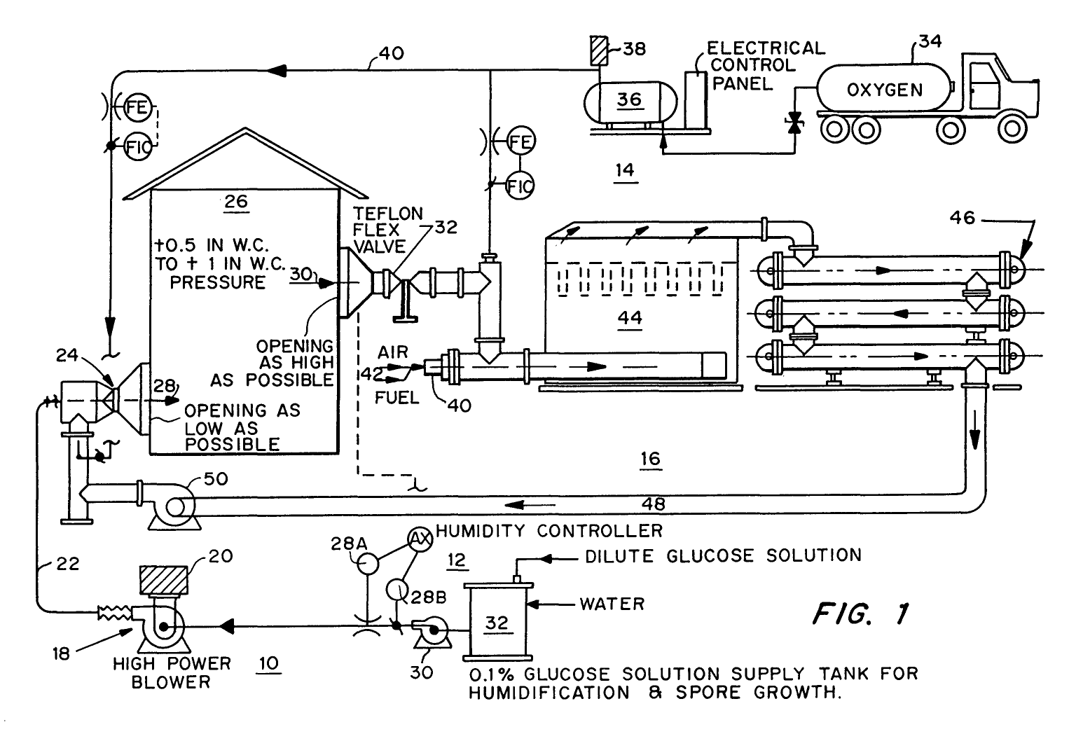

The system is shown in FIG. 1. In a preferred embodiment, the modules are mounted on flat bed trucks. There are three principle components: air intake module 10 including humidity controller 12, ozone generator module 14, and disposal module 16.

The air intake module 10 includes a high power blower 18, preferably a silencer 20, and connections 22 and valving 24 into a sealed contaminated building 26. The intake 28 into the building 26 should be as low as possible, while the outflow 30 should be as high as possible. The blower 18 is preferably connected to the humidity controller 12 by means of valving 28a and 28b connecting to a pump 30 regulating water flow from a tank 32. The tank 32 may contain water or in some embodiments, a dilute glucose (0.1%) or other solution 34 to initiate spore development.

The ozone generator 14 is generated using an electrical control panel to feed oxygen from a tank truck 34 into the generator 36 were the ozone is generated by the ozone destruct unit 38. The ozone is pumped through connections 40 into the inlet 28 via valving 24.

The humidified air is pumped through valving 24 into the sealed contaminated building 26, preferably to between +0.5 and +1 in W.C. pressure. The air exits the building 26 through outflow 30, regulated by a valve 32.

The ozone-humidified air mixture is discharged through the outflow 30 and valve 32 into a burner 40 for incineration. Air and fuel 42 are injected into the kiln to aid in incineration. The incinerated air is then passed through a high temperature filter 44 followed by exposure to ultraviolet lights 46 (and/or ionizers, not shown) and the air recirculated through connections 48 to a blower 50 back into the valving 24 and inlet 28 into the building.

Procedure for Disinfection

In an initial step, the building layout and total volume are determined. All windows, doors, other openings and vents on the building are securely sealed to prevent any air leakage. The ozone flow inlet and outlet is then position so that the gas passage will cover all areas in the building. It is important to be sure that sufficient ozone will be supplied to the air intake of the HVAC system. If the building is equipped with central air conditioning, the system should be turned on. The discharge of the ozone generator should be connected to the selected inlet, which is properly funneled for the connection to a flexible hose. The intake of the scrubber system is connected to a selected outlet for the building. The ozone generator is energized and the blower operated to inject ozone into the building. The amount of ozone is metered to insure the building is fully filled with ozone gas. The ozone is retained in the building for a sufficient time to inactivate the bioagent. This time will typically be longer if the agent is in the form of spores rather than bacteria. The spent ozone is then evacuated from the building by energizing the induced draft fan, which conveys the gas through the scrubbers and vents the treated gas into the atmosphere. Seals on all building openings are removed.

Ozone Generation

Ozone cannot be stored or transported because of its unstable tendency to break down quickly, so ozone must be generated on site. Ozone may be generated by any convenient method, such as corona discharge or UV irradiation, applied to an air, oxygen, or oxygen-enriched air stream, as described for example in U.S. Pat. No. 5,855,856 to Karlson and No. 5,766,560 to Cole (corona discharge) and in U.S. Pat. No. 4,517,084 to Pincon, No. 4,329,212 to Obenshain, No. 4,427,636 to Obenshain, and No. 4,317,044 to Vaseen (UV irradiation).

The system is maintained at the site until an independent testing authority certifies the absence of the specific bacteria, virus, molds, or spores for which this disinfection process is applied. In the event that the test is positive, the disinfection process is repeated until the area is certified for absence of the specific microorganisms.

Conditions that Influence Ozone Disinfection

Preferably, the ozone concentrations is in the range of 1-100 PPM. More preferably, the ozone concentration is in the range of 6-10 PPM. In the most preferred embodiment of the claimed processes, the ozone concentration in the treated area is maintained at 10 PPM for an effective time. Ozone at levels above 1 PPM is considered hazardous to humans. A safe level of residual ozone is considered to be that of ambient, unpolluted, atmospheric air. The U.S. Environmental Protection Agency (EPA) recommends exposure to outdoor air averaging no more than 0.08 PPM ozone for eight hours. Preferably the ozone concentration in the treated area is reduced to ambient concentration, after the area is treated with ozone at the higher disinfecting concentration.

The bactericidal effects of ozone in air can be enhanced through ultrasonication, combination with UVGI (Ultraviolet Germicidal Irradiation) or negative ionization, and optimized humidity level. Lethal to microorganisms, ultraviolet radiation in the range 2250-3020 Angstroms is used in a variety of disinfection applications, a process referred to as Ultraviolet Germicidal Irradiation (UVGI). In a preferred embodiment, the target area is treated with a combination of ozone at a disinfecting concentration and UVGI.

Ozone disinfection can be enhanced by the use of humidified ozone. The ozone penetrates cell wall and membrane of bacteria more easily when the bacteria is coated with a water film which results from the increased humidity level. Studies show that relative humidity of 50% or higher is needed to enhance ozone disinfection, while relative humidity in the range of 60-75% is often used. In a preferred embodiment, the target area is treated with ozone at disinfecting concentration in the relative humidity at 60-75%.

The temperature and air pressure in the area can also be regulated by the system. Temperature is important for the decay of ozone. Air pressure may be important to control the flow rate of the air circulated within the area and the system.

A preferred embodiment is summarized as follows:

The process consists of sealing the area to be treated and illuminating the interior with ultraviolet lights. The sealed area is connected to the system and airflow is established from the sealed area through the system and back to the sealed area. This air flow is maintained until the required concentration of ozone in a controlled humidification environment is reached, and sustained over a certain specified time. The air from the sealed area is sucked, humidified under slight negative pressure and subjected to ozone and oxygen or air mixture to achieve ozone concentration in the air flow of about 10 PPM over a reaction time of about two seconds. Ozonized air is then filtered through dual high efficiency filter candles washed continuously. Humidification sprays use this filter drain liquid. The exhaust from the filter is then subjected to a series of full coverage ultraviolet light for a period of about one second and then subjected to ozone/oxygen or ozone/air mixture to achieve an ozone concentration in the air flow of about 10 PPM in the air returning to the sealed area. This process is continued until the ozone concentration in the air exiting the sealed area is about 10 PPM for an average period of sixty minutes. On achieving this disinfecting period, all of the ozone/oxygen or ozone/air mixture is gradually reduced to an average of 1 PPM over a period of about six minutes. At this point, the ozone/oxygen or ozone/air mixture is turned off and air samples taken to determine the absence of any viable airborne microorganisms and the air certified disinfected. After this successful certification, all of the air exiting the duct fitted with ultra violet lights was incinerated in a kiln. The average kiln temperature over a three second retention time was regulated to exceed 572° F. and the kiln exhaust exited the stack via a wet electrostatic precipitor to control any emissions from the sealed area.

While stabilizing the incineration process, the sealed area was gradually diluted with fresh air until the air exiting the sealed area indicated an ozone concentration to be 0.1 to 0.2 PPM and the exhaust continuously incinerated. On reaching the safe ozone concentration, independent testing company personnel enter the sealed area and take samples, as they deem necessary, to render the facility disinfected. Once the facility is certified disinfected, the system is taken apart under controlled conditions and each segment tested for any deposit for the specific microorganisms. All suspect items are placed in a chamber and treated with humidified ozone mixture with ultraviolet light to render the items inert. Filter medium is then incinerated in the kiln.

We claim:

1. A process for disinfecting an area, the process comprising
connecting a sealed area with a disinfecting system capable of generating ozone, and
circulating air within the sealed area and the system for a time at a concentration of ozone in the range of one to 100 ppm ozone at a relative humidity of 50% or higher.

2. The process according to claim 1 wherein the time is longer than 60 minutes.

3. The process according to claim 1 further comprising reducing the ozone concentration to an ambient concentration.

4. The process according to claim 1 further comprising
illuminating the area and/or the air circulated from the area with Ultraviolet light, ultrasonic energy or negative ionization.

5. The process according to claim 4 wherein the air is illuminated with ultraviolet light having a wavelength in the range of 2250-3020 Angstroms.

6. The process according to claim 1 further comprising adjusting the relative humidity to at least 50%.

7. The process according to claim 6 further comprising adjusting the relative humidity to the range of 60-75%.

8. The process according to claim 1 further comprising incinerating in a kiln air circulated from the area when the ozone concentration is reduced, wherein the kiln is fitted with an emission control system.

9. The process according to claim 7 wherein the average kiln temperature is higher than 572° F.

10. The process according to claim 1 wherein the disinfecting concentration is in the range of six to ten PPM.

11. The process according to claim 1 further comprising measuring the results of disinfecting.

12. The process according to claim 1 further comprising measuring and controlling temperature within the area.

13. The process according to claim 1 further comprising measuring and controlling air pressure within the area.

14. A system for disinfecting an area, the system comprising,
a generator for generating ozone,
a detector for measuring the concentration of ozone, and
a regulator for controlling the generator,
wherein a disinfecting concentration of ozone of between one and 100 ppm and relative humidity of 50% or greater can be achieved and maintained within the area.

15. The system according to claim 14 further comprising
a ventilator for circulating air within the area and the system when they are connected and sealed.

16. The system according to claim 14 further comprising
an UV irradiator for illuminating the area and/or the air circulated from the area with ultraviolet light.

17. The system according to claim 12 further comprising a regulator adjusting humidity within the area.

18. The system according to claim 12 further comprising a kiln for incinerating air circulated from the area.

19. The system according to claim 16 wherein the kiln is fitted with an emission control system.
